(19) United States
(12) Patent Application Publication
(10) Pub. No.: US 2008/0243019 A1
(43) Pub. Date: Oct. 2, 2008

(54) Respiratory function measuring equipment and storage medium

(75) Inventors: Shuko Tsujimura, Tokyo (JP); Hidetoshi Nakamura, Tokyo (JP); Akitoshi Ishizaka, Tokyo (JP); Isao Sato, Yokohama-shi (JP)

Correspondence Address: Edwards Angell Palmer & Dodge LLP, P.O. Box 55874, Boston, MA, 02205, US

(73) Assignee: KEIO University, Tokyo (JP)

(21) Appl. No.: 12/002,767

(22) Filed: Dec. 19, 2007

(30) Foreign Application Priority Data
Dec. 21, 2006 (JP) 2006-344008

(51) Int. Cl. A61B 5/08

(52) U.S. Cl. 600/534; 600/529

(57) ABSTRACT

A respiratory function measuring device comprises: a three-dimensional measuring unit that measures a chest movement and an abdomen movement of a breathing animal; a first measuring unit that measures a time T1 where a rate of volume decrease of the abdomen is maximized in an expiration; a second measuring unit that measures a time T2 where a rate of volume decrease of the chest is maximized in the expiration; and a respiratory time difference outputting unit that computes and outputs a value Tde corresponding to T2-T1. This allows measuring respiratory function to diagnose an obstructive pulmonary disease, a restrictive pulmonary disease, and the like in a natural state, for a subject of a breathing animal, even if the subject does not have a sense of self-awareness.

### BACKGROUND ART

1. Technical Field

The present invention relates to a respiratory function measuring device that measures respiratory function to diagnose an obstructive pulmonary disease, a restrictive pulmonary disease, and the like, and a respiratory function measuring device that can measure respiratory function in a natural state, for a subject of a breathing animal (including a human being in this specification), even if the subject does not have a sense of self-awareness.

2. Description of the Related Art

For a conventional respiratory function measuring device, spirometry has been exclusively used, however, in this test, it is necessary to demand that a patient make his/her best effort to breath while holding a mouthpiece with a nose clip. Therefore, it has been difficult to conduct testing for an infant, an aged person, and a patient with respiratory failure, and it has also been pointed out that the results greatly differ depending on the skill level of the medical technician. Moreover, basic indicators of respiratory function could also not be tested under a natural state.

Moreover, there is an art, in which a three-dimensional measuring device that projects a lighting pattern onto a subject and picks up an image from an angle different therefrom is used to obtain a respiratory waveform of the subject by use of a movement of the lighting pattern according to breathing of the patient (see Patent Document 1, for example).

Further, there is an art, in which the above-mentioned three-dimensional measuring device is used to obtain respective respiratory waveform patterns of the chest and abdomen (see Patent Document 2, for example).

[Patent Document 1] Japanese Unexamined Patent Application Publication No. 2002-175582

[Patent Document 2] Japanese Unexamined Patent Application Publication No. 2005-246033

### SUMMARY OF THE INVENTION

However, these arts that use three-dimensional measuring devices to obtain respiratory waveforms are provided for the purpose of detecting abnormal breathing of great urgency, and thus not ones for accurately measuring the function of respiratory organs such that a respiratory disease can be diagnosed, and application for diagnosis of a respiratory disease has not been considered.

In view of the abovementioned problems, it is an object of the present invention to provide a respiratory function measuring device that can measure respiratory function to diagnose an obstructive pulmonary disease, a restrictive pulmonary disease, and the like in a natural state, for a subject of a breathing animal, even if the subject does not have a sense of self-awareness and a storage medium.

A respiratory function measuring device of the present invention comprises: a three-dimensional measuring means that measures a chest movement and an abdomen movement of a breathing animal; a first measuring means that measures a time T1 where a rate of volume decrease of the abdomen is maximized in an expiration; a second measuring means that measures a time T2 where a rate of volume decrease of the chest is maximized in the expiration; and a respiratory time difference outputting means that computes and outputs a value Tde corresponding to T2-T1.

Moreover, the respiratory time difference outputting means computes Tde in terms of multiple expirations and computes and outputs a value Av(Tde) corresponding to an average value thereof, whereby the value Av (Tde) can be provided as a stable indicator.

Moreover, a respiratory function measuring device of the present invention comprises: a three-dimensional measuring means that measures a chest movement and an abdomen movement of a breathing animal; a third measuring means that measures a time T3 where a rate of volume increase of the abdomen is maximized in an inspiration; a fourth measuring means that measures a time T4 where a rate of volume increase of the chest is maximized in the inspiration; and a respiratory time difference outputting means that computes and outputs a value Tdi corresponding to T4-T3.

Moreover, the respiratory time difference outputting means computes Tdi in terms of multiple inspirations and computes and outputs a value Av(Tdi) corresponding to an average value thereof, whereby the value Av (Tdi) can be provided as a stable indicator.

Moreover, a respiratory function measuring device of the present invention comprises: a three-dimensional measuring means that measures a body movement of a breathing animal; a fifth measuring means that measures an inspiration time T1 of a respiration; a sixth measuring means that measures an expiration time Te of the respiration; and a respiratory ratio outputting means that computes and outputs a value R corresponding to Ti/Te.

Moreover, the respiratory ratio outputting means measures R in terms of multiple respirations and computes and outputs a value Av(R) corresponding to an average value thereof, whereby the value Av(R) can be provided as a stable indicator.

Moreover, a respiratory function measuring device comprises: a three-dimensional measuring means that measures a body movement of a breathing animal; and a respiratory minute volume outputting means that outputs a value corresponding to a respiratory minute volume.

Moreover, the present invention provides a computer-readable storage medium having a program recorded thereon where the program makes a computer as the above-mentioned respiratory function measuring device.

Patients with obstructive ventilatory impairment include all age brackets from infants to the elderly, and the number of domestic patients is considered to be more than 10 million even only in terms of chronic obstructive pulmonary disease and bronchial asthma. Diagnosis thereof has exclusively relied on spirometry by forced expiration. It is therefore considered that there are many cases of chronic and irreversible decline in lung function caused without being diagnosed as such a disease. According to the present invention, a large-scaled screening of respiratory function is enabled without a burden placed on either the patient or health professionals, so that detection of a case of a decline in lung function, follow-up, and therapy evaluation can be considerably easily carried out.

The present specification includes the contents described in the specification and/or drawings of Japanese Patent Application No. 2006-344008, which forms the basis of the priority right of the present application.

### BRIEF DESCRIPTION OF THE DRAWINGS

FIG. 1 is a view showing an outline of the configuration of a respiratory function measuring device according to Example 1.

FIG. 2A and FIG. 2B are graphs for explaining principles of the invention of Example 1.

FIG. 3 is a view of a comparison, between a COPD patient and a healthy person, of a delay of the chest from the abdomen in the maximum volume decrease time of expiration in quiet breathing state.

FIG. 4 is a view of a comparison, between before and after use of an inhalation, of a delay of the chest from the abdomen in the maximum volume decrease time of expiration in quiet breathing state.

FIG. 5A and FIG. 5B are graphs for explaining principles of the invention of Example 2.

FIG. 6 is a view of a comparison, between a COPD patient and a healthy person, of the inspiration time/expiration time in quiet breathing state.

FIG. 7 is a view of a comparison, between before and after use of an inhalation, of the inspiration time/expiration time in quiet breathing state.

FIG. 8 is a view of a comparison, between a COPD patient and a healthy person, of the respiratory minute volume in quiet breathing state.

FIG. 9 is a view of a comparison, between before and after use of an inhalation, of the respiratory minute volume in quiet breathing state.

### DETAILED DESCRIPTION OF THE INVENTION

Hereinafter, embodiments for carrying out the present invention will be described in detail with reference to the accompanying drawings.

### Example 1

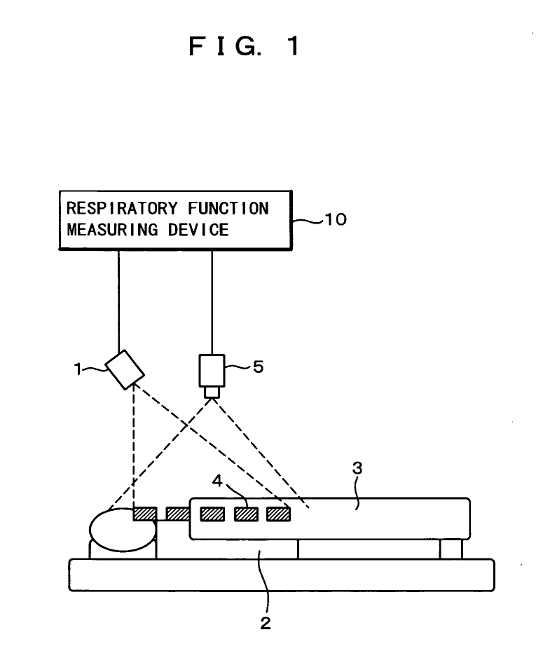

FIG. 1 is a view showing an outline of the configuration of a respiratory function measuring device according to Example 1 of the present invention. The respiratory function measuring device 10 includes a body, a lighting pattern projection means 1, and an image pickup means 5. First, a lighting pattern 4 is projected from the lighting pattern projection means 1 onto a body 2 of a sleeper or bedding 3. The wavelength of projecting light is preferably set to that of infrared rays because the sleeper needs not be aware of being monitored. The lighting pattern 4 projected onto the body 2 or the bedding 3 is picked up continuously as an image by the image pickup means 5. The image pickup means 5 can pick up an image of infrared rays, which correspond to the wavelength of the projected light. Due to a movement in the optical axis direction of the image pickup means 5 of the body 2 or the bedding 3 resulting from a movement of the body 2, a shift of the lighting pattern having a different optical axis therefrom occurs within an imaging plane, and a waveform corresponding to this shift of the lighting pattern is obtained as a respiratory waveform from the image picked up by the image pickup means 5. For determining the respiratory minute volume (the amount of air that enters and exits the lungs), the size (that is, amplitude) of an obtained respiratory waveform (that is, a vertical motion waveform of the body surface) is calibrated based on the results of measurements simultaneously conducted with a person of a similar figure using spirometry and the respiratory function measuring device of the present invention.

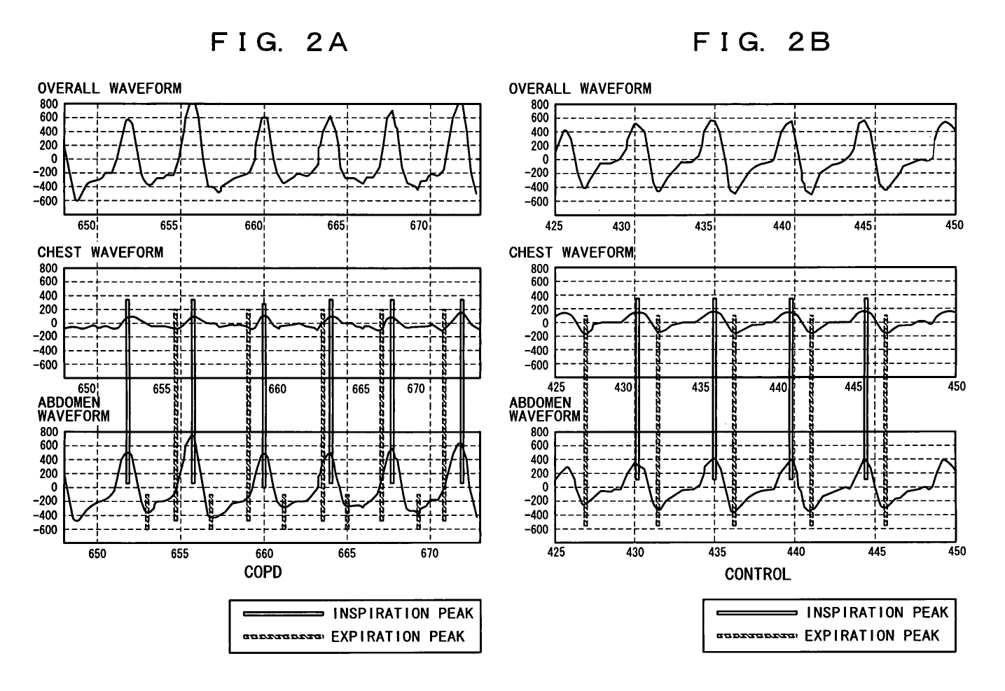

FIG. 2A and FIG. 2B are graphs for explaining principles of the invention of Example 1. These show respiratory rate waveforms plotted with a respiratory rate with an arbitrary scale by differentiating a respiratory waveform on the vertical axis and time with an arbitrary scale on the horizontal axis. On the left side, shown are an overall waveform, a chest waveform, and an abdomen waveform of a respiratory rate from the top in the case of a COPD (chronic obstructive pulmonary disease) patient. On the right side, shown are likewise an overall waveform, a chest waveform, and an abdomen waveform of a respiratory rate from the top in the case of a healthy person used as a control. The chest waveform is a waveform obtained from a chest image that has been picked up. The abdomen waveform is a waveform obtained from an abdomen image that has been picked up. The overall waveform is a waveform obtained by synthesizing a chest waveform and an abdomen waveform, that is, by averaging both waveforms.

The positive peaks, that is, inspiration peaks denoted with thick solid lines from the chest waveform to the abdomen waveform, that is, times of the highest inspiration rates are the same between the chest and abdomen in terms of either the COPD patient or control. On the other hand, the negative peaks, that is, expiration peaks denoted with thick dotted lines from the chest waveform to the abdomen waveform, that is, times of the highest expiration rates are the same between the chest and abdomen in terms of the control, but in terms of the COPD patient, the times are delayed in the chest from the abdomen. It is therefore considered that an obstructive pulmonary disease can be diagnosed by computing and outputting (T2-T1: where T2 is a time when the rate of volume decrease of the chest is maximized in expiration, T1 is a time when the rate of volume decrease of the abdomen is maximized in expiration.)

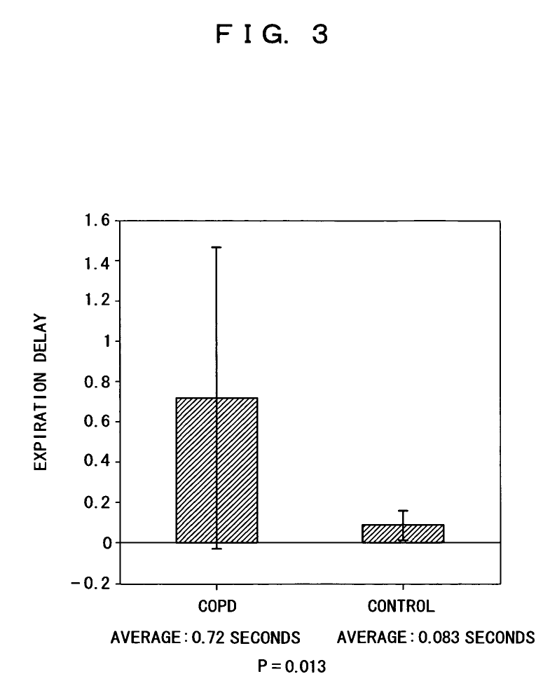

FIG. 3 is a view of a comparison, between a COPD patient and a healthy person, of a delay of the chest from the abdomen in the maximum volume decrease time of expiration in quiet breathing state. Here, the vertical axis represents delay time (second). An average value of 12 COPD patient samples was 0.72 seconds, while an average value of 10 control samples was 0.083, and thus there is a significant difference with a value, P=0.013.

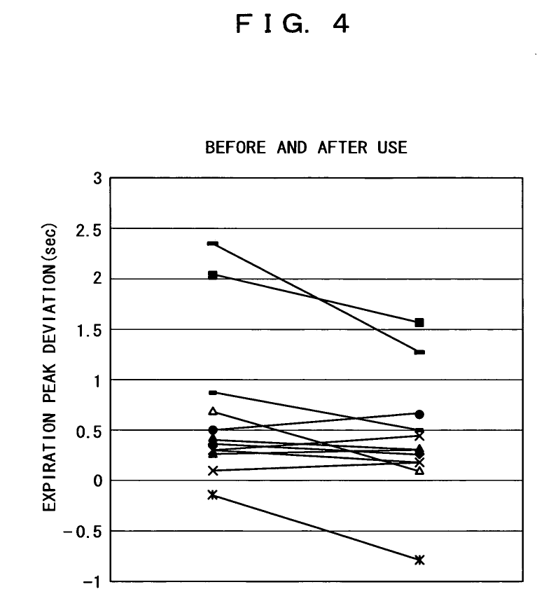

FIG. 4 is a view of a comparison, between before and after use of an inhalation, of a delay of the chest from the abdomen in the maximum volume decrease time of expiration in quiet breathing state. Here, the vertical axis represents delay time (second). The time of a delay of the chest from the abdomen in the maximum volume decrease time of expiration, before and after (6 to 12 weeks) an intake of a bronchodilator (tiotropium), of 12 COPD patients was: 0.72 seconds on average before use (left side); and 0.46 seconds on average after use (right side), with a P value of P=0.036. A reduction in delay time due to a bronchodilator intake was thus recognized with a significant difference.

Based on the above, it is obvious that the (T2-T1) is meaningful as an indicator to diagnose an obstructive pulmonary disease.

Moreover, by analogy of this, with regard to a restrictive pulmonary disease, (T4-T3: where T4 is a time when the rate of volume increase of the chest is maximized in expiration, T3 is a time when the rate of volume increase of the abdomen is maximized in expiration) can be used as an indicator for diagnosis.

As a matter of course, these times can be provided as stable indicators by averaging in terms of multiple respirations.

Due to these indicators, a large-scaled screening of respiratory function is enabled without a burden placed on either the patient or health professionals, so that detection of a case of a decline in lung function, follow-up, and therapy evaluation can be considerably easily carried out.

### Example 2

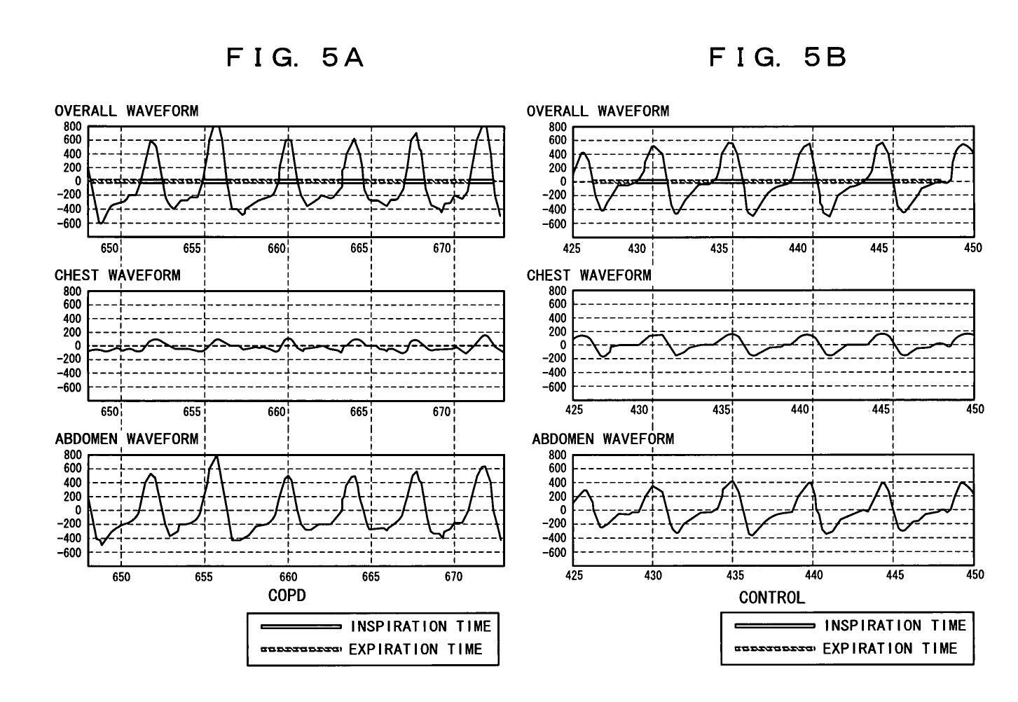

FIG. 5A and FIG. 5B are graphs for explaining principles of the invention of Example 2. The graphs are the same as those of Example 1. In Example 2, attention is focused on a ratio of inspiration time and expiration time within a respiratory time. Because each graph shows a respiratory rate waveform, a positive time of the waveform indicates an inspiration time and a negative time indicates an expiration time. On the horizontal axis of an overall waveform, shown is an inspiration time by a thick solid line, and an expiration time by a thick dotted line. It can be understood that a COPD patient has a longer fraction of expiration time as compared with a control. It is therefore considered that an obstructive pulmonary disease can be diagnosed by computing and outputting (an inspiration time/an expiration time.)

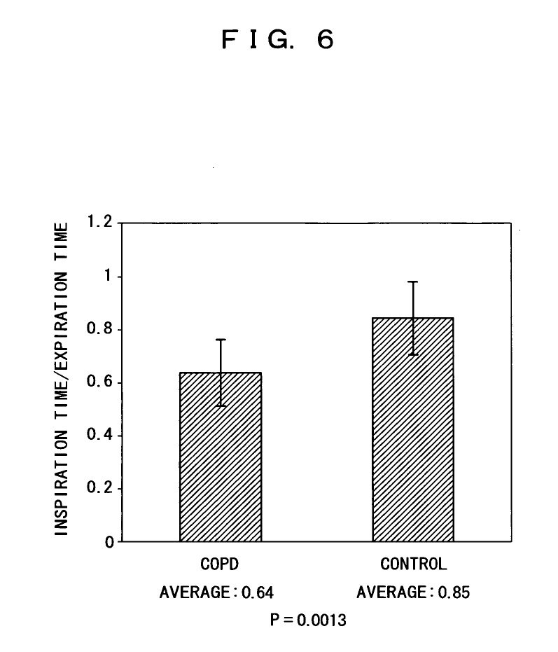

FIG. 6 is a view of a comparison, between a COPD patient and a healthy person, of the inspiration time/expiration time in quiet breathing state. Here, the vertical axis represents an inspiration time/expiration time. An average value of 12 COPD patient samples was 0.64, while an average value of 10 control samples was 0.85, and thus there is a significant difference with a P value, P=0.0013.

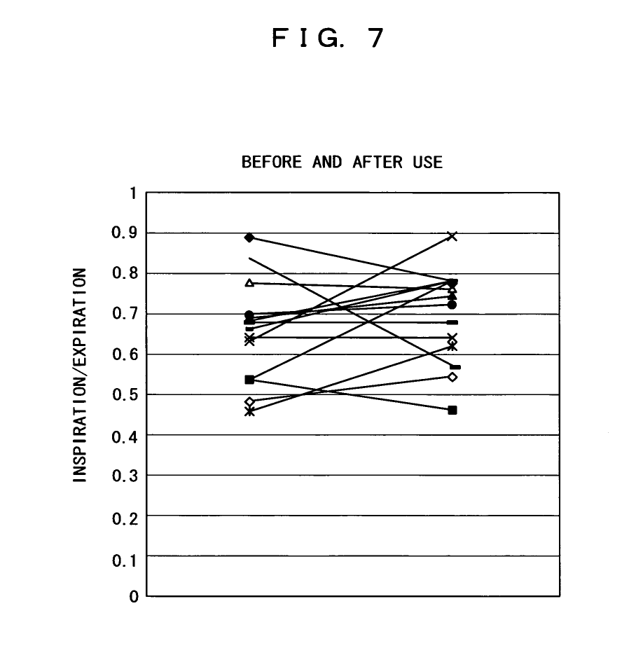

FIG. 7 is a view of a comparison, between before and after use of an inhalation, of the inspiration time/expiration time in quiet breathing state. Here, the vertical axis represents an inspiration time/expiration time. The inspiration time/expiration time, before and after (6 to 12 weeks) an intake of a bronchodilator (tiotropium), of 12 COPD patients was: 0.64 on average before use (left side); and 0.70 on average after use (right side), with a P value of P=0.106. An obvious increase in inspiration time/expiration time due to a bronchodilator intake was thus recognized.

Based on the above, it is obvious that the inspiration time/expiration time is meaningful as an indicator to diagnose an obstructive pulmonary disease.

As a matter of course, this inspiration time/expiration time can be provided as a stable indicator by averaging in terms of multiple respirations.

Due to this indicator, a large-scaled screening of respiratory function is enabled without a burden placed on either the patient or health professionals, so that detection of a case of a decline in lung function, follow-up, and therapy evaluation can be considerably easily carried out.

### Example 3

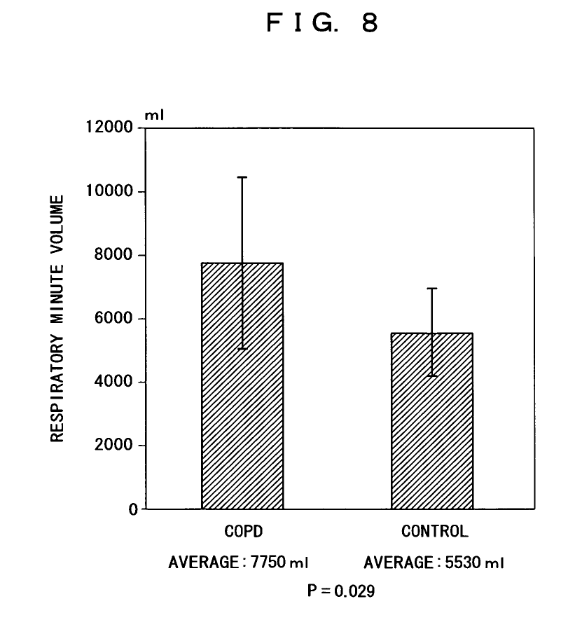

FIG. 8 is a view of a comparison, between a COPD patient and a healthy person, of the respiratory minute volume in quiet breathing state. Here, the vertical axis represents a respiratory minute volume (ml). The respiratory minute volume corresponds to an amount of ventilation per one minute. An average value of 12 COPD patient samples was 7750 ml, while an average value of 10 control samples was 5530 ml, and thus there is a significant difference with a P value, P=0.029. It is therefore considered that an obstructive pulmonary disease can be diagnosed by computing and outputting a respiratory minute volume. The respiratory minute volume can be determined by calculating the amount of one ventilation×the respiratory rate (times/minute). The amount of one ventilation can be determined, as described above, by calibrating the size of a respiratory waveform according to a spirometry measurement.

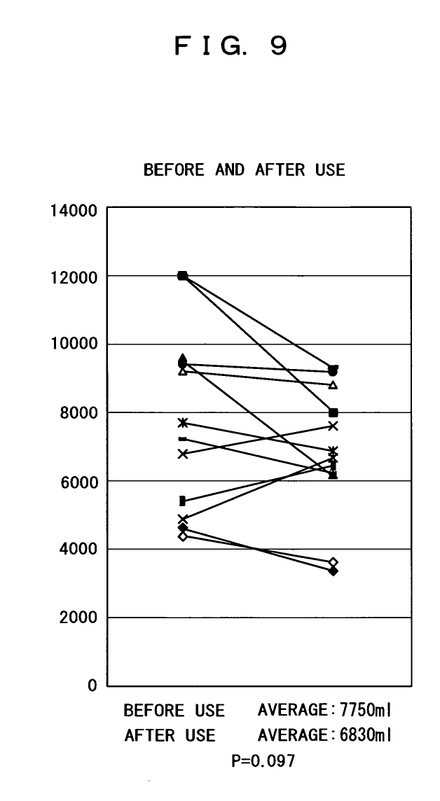

FIG. 9 is a view of a comparison, between before and after use of an inhalation, of the respiratory minute volume in quiet breathing state. Here, the vertical axis represents a respiratory minute volume (ml). The respiratory minute volume, before and after (6 to 12 weeks) an intake of a bronchodilator (tiotropium), of 12 COPD patients was: 7750 ml on average before use (left side); and 6830 ml on average after use (right side), with a P value of P=0.097. A reduction in respiratory minute volume due to a bronchodilator intake was thus recognized.

Based on the above, it is obvious that the respiratory minute volume is meaningful as an indicator to diagnose an obstructive pulmonary disease.

Due to this indicator, a large-scaled screening of respiratory function is enabled without a burden placed on either the patient or health professionals, so that detection of a case of a decline in lung function, follow-up, and therapy evaluation can be considerably easily carried out.

However, the present invention is not limited to the abovementioned examples.

A respiratory function measuring device of the present invention can also be realized by a program to operate a computer as the present respiratory function measuring device. This program may be stored in a storage medium that can be read by a computer.

This storage medium recorded with the program may be a ROM itself of the respiratory function measuring device 10 shown in FIG. 1, or may be a storage medium such as a CD-ROM that can be read, when a program reading device such as a CD-ROM drive is provided as an external storage device, by inserting therein the storage medium.

Moreover, the abovementioned storage medium may be a magnetic tape, a cassette tape, a flexible disk, a hard disk, an MO/MD/DVD or the like, or a semiconductor memory.

All publications, patents and patent applications cited herein are hereby incorporated by reference in their entirety.

What is claimed is:

1. A respiratory function measuring device comprising:
a three-dimensional measuring means that measures a chest movement and an abdomen movement of a breathing animal;
a first measuring means that measures a time T1 where a rate of volume decrease of the abdomen is maximized in an expiration;
a second measuring means that measures a time T2 where a rate of volume decrease of the chest is maximized in the expiration; and
a respiratory time difference outputting means that computes and outputs a value Tde corresponding to T2-T1.

2. The respiratory function measuring device according to claim 1, wherein the respiratory time difference outputting means computes Tde in terms of multiple expirations and computes and outputs a value Av(Tde) corresponding to an average value thereof.

3. A respiratory function measuring device comprising:
a three-dimensional measuring means that measures a chest movement and an abdomen movement of a breathing animal;
a third measuring means that measures a time T3 where a rate of volume increase of the abdomen is maximized in an inspiration;
a fourth measuring means that measures a time T4 where a rate of volume increase of the chest is maximized in the inspiration; and
a respiratory time difference outputting means that computes and outputs a value Tdi corresponding to T4-T3.

4. The respiratory function measuring device according to claim 3, wherein the respiratory time difference outputting means computes Tdi in terms of multiple inspirations and computes and outputs a value Av(Tdi) corresponding to an average value thereof.

5. A respiratory function measuring device comprising:
a three-dimensional measuring means that measures a body movement of a breathing animal;
a fifth measuring means that measures an inspiration time Ti of a respiration;
a sixth measuring means that measures an expiration time Te of the respiration; and
a respiratory ratio outputting means that computes and outputs a value R corresponding to Ti/Te.

6. The respiratory function measuring device according to claim 5, wherein the respiratory ratio outputting means measures R in terms of multiple respirations and computes and outputs a value Av(R) corresponding to an average value thereof.

7. A respiratory function measuring device comprising:
a three-dimensional measuring means that measures a body movement of a breathing animal; and
a respiratory minute volume outputting means that outputs a value corresponding to a respiratory minute volume.

8. A computer-readable storage medium having a program recorded thereon where the program makes a computer as the respiratory function measuring device according to claim 1.

9. A computer-readable storage medium having a program recorded thereon where the program makes a computer as the respiratory function measuring device according to claim 3.

10. A computer-readable storage medium having a program recorded thereon where the program makes a computer as the respiratory function measuring device according to claim 5.

11. A computer-readable storage medium having a program recorded thereon where the program makes a computer as the respiratory function measuring device according to claim 7.
